(19) United States
(12) Patent Application Publication
(10) Pub. No.: US 2020/0114242 A1
(43) Pub. Date: Apr. 16, 2020

(54) CYCLIST RELATIVE PERFORMANCE MEASUREMENT APPARATUS

(71) Applicant: Garmin Switzerland GmbH, Schaffhausen (CH)

(72) Inventors: Kenneth A. Carlson, Rochester, MN (US); Ross G. Stirling, Cochrane (CA); Theodorus J. Smit, Calgary (CA)

(21) Appl. No.: 16/653,370

(22) Filed: Oct. 15, 2019

Related U.S. Application Data
(60) Provisional application No. 62/746,070, filed on Oct. 16, 2018.

(51) Int. Cl. A63B 71/06; A63B 69/16; B62J 11/00; B62J 99/00; A63B 24/00

(52) U.S. Cl. CPC A63B 71/0622; A61B 5/1123; A63B 69/16; B62J 11/00; B62J 99/00; A63B 24/0062; A63B 2220/40; A63B 2220/833; A63B 2220/836; A63B 2220/51; A63B 2220/12; A63B 2071/0694; A63B 2220/73; A63B 2071/0691; A63B 2225/50; B62J 2099/0013; B62J 2099/0033; A63B 71/0686

(57) ABSTRACT

Techniques are disclosed to enable relative performance measurement of a cyclist by way of an electronic device for use in connection with a bicycle. The electronic device determines and communicates an exertion level and a corresponding actual performance metric achieved by a cyclist based on movement characteristics of the bicycle and whether the cyclist's shoes are clipped into pedals on the bicycle. The device uses sensors for determining the movement characteristics of the bicycle in connection with a memory element and a processing element. The processing element ascertains a pedal clip status event based on the movement characteristics of the bicycle, and the device displays information about the exertion level of the cyclist as correlated with the actual bicycle performance metrics.

### RELATED APPLICATIONS

The current U.S. non-provisional patent application claims priority benefit with regard to all common subject matter of an earlier-filed U.S. provisional patent application titled “METHOD AND APPARATUS FOR DETERMINING CYCLIST FOOT STATUS”, Application Ser. No. 62/746,070, filed Oct. 16, 2018. The earlier-filed application is hereby incorporated by reference into the current application in its entirety.

### BACKGROUND

In the context of mountain bike riding and cyclocross racing, cyclists frequently endeavor to improve their relative performance. Specifically, a cyclist may repeat a particular course on multiple occasions to determine whether her or she can complete the course with greater mastery, in terms of parameters such total time for completion of the course as well as speed, cleanness of the ride, i.e. percentage of time pedaling the bike while clipped in as opposed to clipped out and walking the bike. Both recreational and competitive cyclists desire knowledge regarding how their performance compares to other riders on the same course. Traditionally, a test to determine relative competitiveness would take place in the form or a race or cyclocross competition, wherein riders line up for shoulder-to-shoulder racing or time trials on a particular course. To become more competitive, cyclists require mechanisms to measure and compare relative performance. Accordingly, there is a need for systems to provide this information to cyclists.

Bicycles may be configured to measure a cyclist's level of effort. For example, a bicycle may include a pedal with a pedal spindle provided with one or more sensors configured to measure the forces exerted by the cyclist on the pedal. More specifically, Garmin's Vector™ pedals incorporate a plurality of sensors that measures the forces applied by a cyclist to a bicycle's pedals based on an amount of deformation of the bicycle's pedal spindles. This technology is described in U.S. Pat. No. 8,011,242, which is hereby incorporated by reference into the current application in its entirety. The sensors are coupled with a memory element configured to store executable instructions, and a processing element configured to execute those instructions in order to analyze the measured forces and provide information related to the pedaling of the cyclist. The sensors and/or processing element may be coupled with a display configured to communicate the information to the cyclist. The information may include a visual indication of the determined forces, where forces are being wasted, and where energy can be saved without affecting driving force and speed. Thus, one use for the information is to improve the pedaling efficiency of the cyclist by reducing wasted force exerted on the pedal.

### SUMMARY

Techniques are disclosed to enable relative performance measurement of a cyclist by way of an electronic device for use in connection with a bicycle having gears, a gear selector, handlebars, and pedals. The electronic device determines and communicates to a user of the device an exertion level and a corresponding actual performance metric achieved by a cyclist based on movement characteristics of the bicycle and whether one or both of the cyclist's shoes are clipped into the pedals. The device has at least one sensor for determining the movement characteristics of the bicycle, at least one memory element configured to store the movement characteristics of the bicycle, at least one processing element coupled with the sensor and the memory element. The processing element ascertains at least one pedal clip status event based on the movement characteristics of the bicycle. Finally, the device displays information about the exertion level of the cyclist and the corresponding actual cycle performance metric.

This summary is provided to introduce a selection of concepts in a simplified form that are further described below in the detailed description. This summary is not intended to identify key features or essential features of the claimed subject matter, nor is it intended to be used to limit the scope of the claimed subject matter. Other aspects and advantages of the present technology will be apparent from the following detailed description of the embodiments and the accompanying drawing figures.

### BRIEF DESCRIPTION OF THE DRAWINGS

The figures described below depict various aspects of the articles of manufacture disclosed herein. It is understood that these figures depict exemplary embodiments and particular aspects of the disclosed articles of manufacture. The exemplary features illustrated in the figures are intended to represent these aspects of the various disclosed embodiments and not intended to limit the claimed scope to any particular feature. Further, whenever possible, the following description refers to the reference numerals included in the figures, in which features depicted in multiple figures are designated with consistent reference numerals.

FIG. 1 is a side elevation view of a bicycle with an electronic device configured to determine a relative performance measurement of a cyclist;

FIG. 2 is a plan view of the electronic device of FIG. 1;

FIG. 3 is a schematic depiction of elements of the electronic device of FIG. 1;

FIG. 4 is a side elevation view of the cyclist with the cyclist's left shoe clipped-in to the left pedal of the bicycle and the cyclist's right shoe clipped-out;

FIG. 5 is a side elevation view of the cyclist with the cyclist's feet clipped out and walking beside the bicycle;

FIG. 6 is a representation of a display of clip-in and clip-out events;

FIG. 7 is a representation of a map display of clip-in and clip-out events on a particular course;

FIG. 8 is a flowchart of steps involved in a method implemented by the electronic device of FIG. 1; and

FIG. 9 is a fragmentary side elevation view of the cyclist's foot exerting force on a pedal component of the bicycle of FIG. 1.

### DETAILED DESCRIPTION

The following text sets forth a detailed description of numerous different embodiments. However, it is understood that the detailed description is to be construed as exemplary only and does not describe every possible embodiment since describing every possible embodiment would be impractical. In light of the teachings and disclosures herein, numerous alternative embodiments may be implemented.

It is understood that, unless a term is expressly defined in this patent application using the sentence “As used herein, the term ‘______’ is hereby defined to mean . . . ” or a similar sentence, there is no intent to limit the meaning of that term, either expressly or by implication, beyond its plain or ordinary meaning, and such term should not be interpreted to be limited in scope based on any statement made in any section of this patent application.

The following detailed description of the technology references the accompanying drawings that illustrate specific embodiments in which the technology may be practiced. The embodiments are intended to describe aspects of the technology in sufficient detail to enable those skilled in the art to practice the technology. Other embodiments may be utilized and changes may be made without departing from the scope of the present technology. The following detailed description is, therefore, not to be taken in a limiting sense. The scope of the present technology is defined only by the appended claims, along with the full scope of equivalents to which such claims are entitled.

In this description, references to “one embodiment”, “an embodiment”, or “embodiments” mean that the feature or features being referred to are included in at least one embodiment of the technology. Separate references to “one embodiment”, “an embodiment”, or “embodiments” in this description do not necessarily refer to the same embodiment and are also not mutually exclusive unless so stated and/or except as will be readily apparent to those skilled in the art from the description. For example, a feature, structure, act, etc. described in one embodiment may also be included in other embodiments, but is not necessarily included. Thus, the present technology may include a variety of combinations and/or integrations of the embodiments described herein.

Embodiments of the technology apply to the field of cyclist performance analysis, and more specifically, to determining relative performance measurement of a cyclist. “Actual bicycle performance” relates to any objective metric regarding performance of the bicycle while in use such as cycle speed over land, uphill or downhill forward progress, power output, total energy produced, or distance of forward progress in a particular racecourse or route. By contrast, “cyclist effort level” or “perceived exertion” relates to heart pulse rate, clip-out events, such as left foot vs. right foot clip-out rate, clip-outs per lap and clip-outs per race as well as other directly or indirectly determinable parameters, and subjective metrics such as cyclist perceived exertion and fatigue level as measured by the need, for example, to downshift or to clip-out and walk the bicycle.

Over the course of a mountain bike ride or cyclocross race, a cyclist may encounter different riding scenarios that require him or her to clip in or out with one or both feet. In a basic scenario, the cyclist is riding on a relatively smooth and stable surface that is either relatively flat or has a slight incline. In this basic scenario, the cyclist will typically have shifted gears into a relatively higher gear and be pedaling at a strong and steady cadence either sprinting, pushing ahead steadily, or recovering, but in any case with such favorable riding conditions, a strong cyclist in the basic scenario will typically be clipped in with both feet and pedaling. In an alternative scenario, a cyclist may stop to take a break to rest and/or eat and drink. In such an alternative scenario, the cyclist may clip out with one or both feet and straddle the frame of the bicycle with one or both feet on the ground. In another scenario, the cyclist may be navigating a descent with both feet clipped in, either seated or standing and either pedaling or coasting. In a significant, relatively high-speed descent, the cyclist may have both feet clipped in. However, in a slower-speed descent over rough terrain, a cyclist may clip out with one or both feet to push off the ground at points of obstacles or rough terrain. Similarly, in an area of rough terrain that is either relatively flat or uphill, the cyclist may clip out with one or both feet to push off the ground to stabilize the bicycle or move it forward.

Embodiments of the present invention utilize one or more pedal-mounted sensors, such as a Vector pedal or any other force and acceleration sensor(s), to determine the status of the cyclist's foot. For example, embodiments of the present invention can determine if the cyclist's foot is on the pedal. In some configurations, cyclist clip-in and clip-out status is determined—e.g., by utilizing one or more sensor measurements from the pedal, a processor in communication with the pedal sensors can determine when the cyclist has engaged the bicycle's pedals with his or her cleats, and therefore determine how the cyclist is moving the bike forward. Thus, these one or more sensors may be utilized to classify the cyclist's riding.

In one configuration, pedal force and acceleration is measured to determine cyclist clip-in and clip-out. For example, the processing element can use force and acceleration to detect when the cyclist's shoe (cleat) disconnects from the pedal. In some configurations, the processor element compares signals from the pedal force and acceleration signals to known signals for “clip-in” and “clip-out” to ascertain cyclist foot status. Additionally, or alternatively, the processor element may utilize a stored model that correlates pedal force and/or acceleration to clip-in and clip-out. This detection can also be accomplished by way of a wearable sensor, for example, a Garmin Foot Pod which is positioned on the cyclist's body, for example, attached to the laces of one or both of the cyclist's shoes. In various embodiments, an accelerometer in the Foot Pod detects an acceleration signature that is consistent with a clip-in or clip-out event. In an alternative embodiment, sensors within a bicycle computer “head” unit such as the Garmin Edge®, which can sense acceleration force and gyroscope signatures that evidence a clip-in or clip-out event by, for example, measuring sharp impacts in connection with handlebar and frame tilt movements that are distinguishable from normal riding or walking conditions.

In another configuration, sensor information is used to determine if a person is walking beside their bicycle. Pedal sensors, or other sensors mounted on a bicycle frame, can detect steps, such as via detecting step acceleration signals, and orientation context can be derived that elucidate whether the bike is being moved in a way that is inconsistent with movement of the bicycle as it is being ridden by a cyclist with one or both feet clipped-in.

Garmin's Vector™ pedals may be used to provide the measurements needed to identify clip-in and clip-out events. More specifically, the plurality of sensors described in U.S. Pat. No. 8,011,242 may be used to measure the radial, tangential, and other forces on the bicycle's pedals to facilitate observing one or more of the identifying features that identify clip-in and clip-out events. The Vector™ pedals themselves may be configured to perform this analysis and/or other devices, such as Garmin's Edge® or Forerunner® global positioning system (GPS) devices, paired with the Vector™ pedals may perform this analysis to identify the events.

In some cases, the geographic locations and time durations of clip-in and clip-out events may be of interest, as well as whether the cyclist is walking and the step cadence while waking in the bicycle. As such, it may be desirable to combine records of clip-in status and clip-out rates with geolocation data such as can be obtained from GPS devices such as Garmin's Edge® or Forerunner® GPS devices. Similarly, in some cases, other bicycle performance metrics, such as speed or attitude, and cyclist performance metrics, such as heart rate, associated with being clipped-in or out and walking or riding may be of interest. As such, it may be desirable to combine statistics regarding clipped-in status and changes with statistics regarding such actual bicycle performance metrics.

When combined with Garmin's Edge® or Forerunner® GPS devices, the cyclist may record their effort level and actual performance data with correlated geographic location data and one or more performance metrics. The relative bicycle performance metric data may be provided to the cyclist in real-time (via the Edge® or Forerunner® GPS devices or other devices such as smart phones) to allow the cyclist to adjust their performance if necessary. Thus, embodiments of the invention may enable the Vector™ pedals (and/or a paired device such as the Edge® or Forerunner® GPS devices) to sense a cyclist's relative performance in real time, display the associated performance metrics in real time, and correlate the performance metrics with GPS location data and/or past performance metrics data associated with the particular cyclist or other riders.

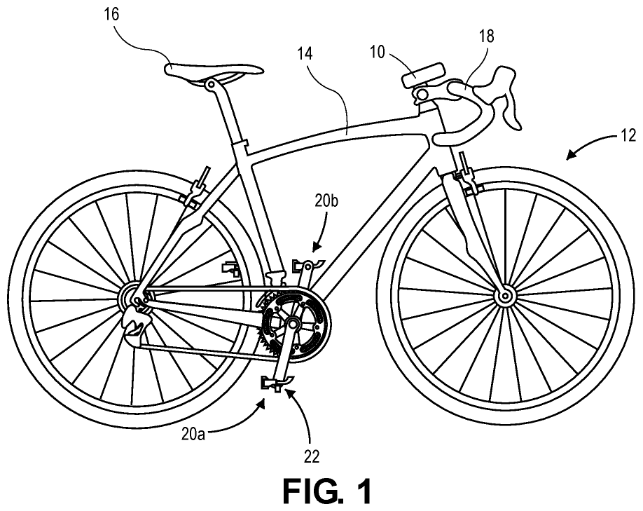

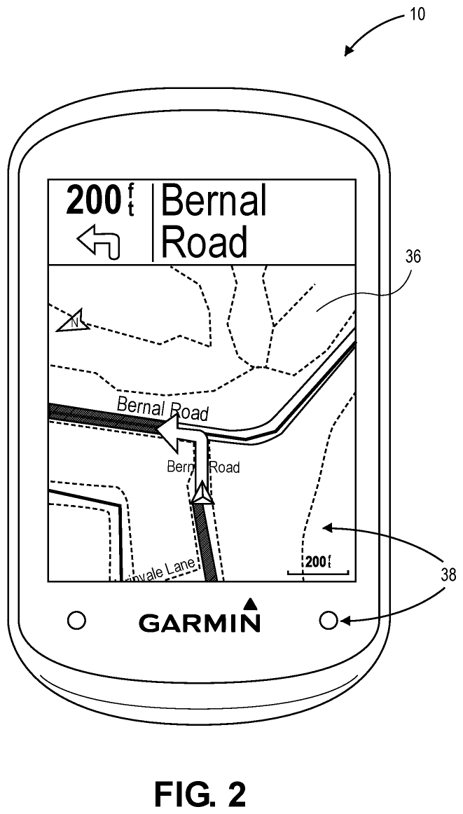

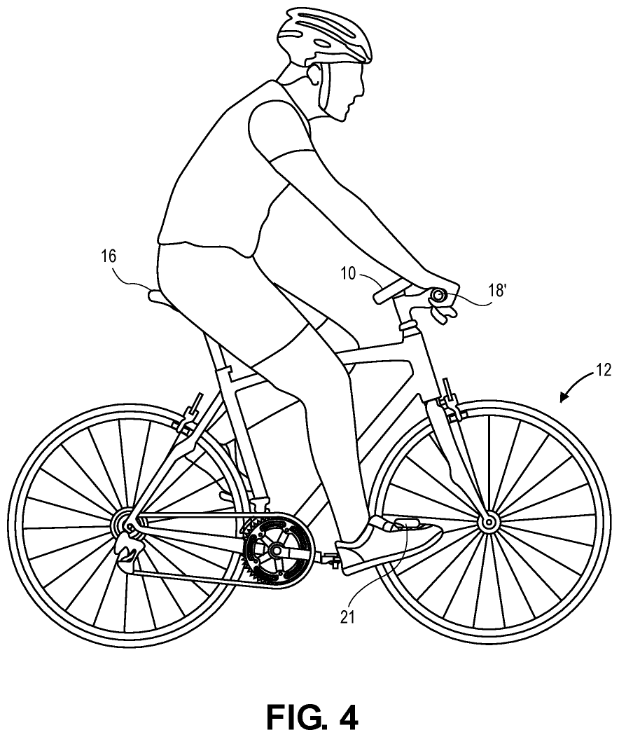

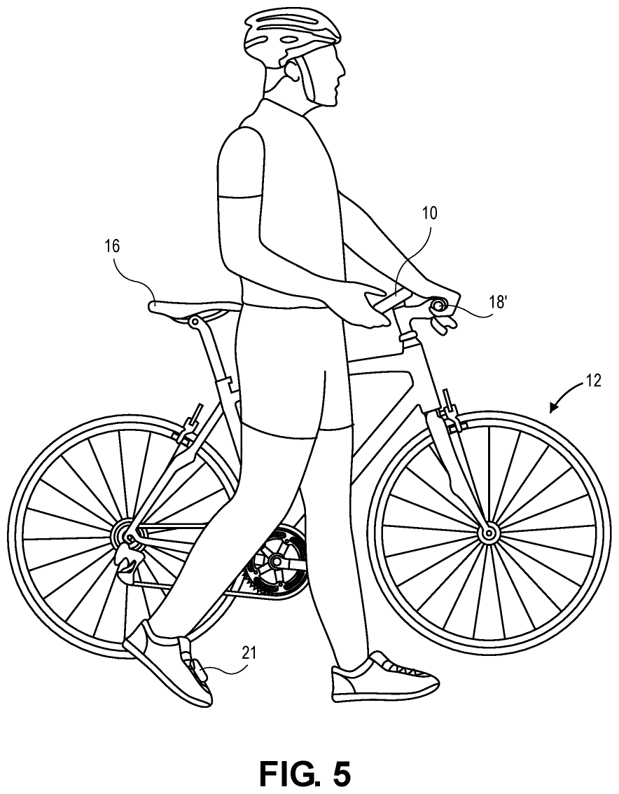

Embodiments of the technology will now be described in more detail with reference to the drawing figures. Referring initially to FIGS. 1 and 2, an electronic device 10 configured to measure bicycle movement characteristics while the cyclist is riding a bicycle 12. The bicycle 12 is shown broadly comprising a frame 14, a seat 16, a handlebar 18, left and right pedals 20 a,20 b, and one or more sensors 22 associated with the left and right pedals 20 a,20 b. The electronic device 10 may be removably mountable on the bicycle 12, and is shown mounted on the handlebar 18 in FIG. 1 and unmounted in FIGS. 2, 6, and 7. The bicycle 12, including the frame 14, seat 16, handlebar 18, and left and right pedals 20 a,20 b may be of substantially any suitable type and design, and any details of these components of the system described herein or shown in the figures are for illustrative purposes only and are not limiting of the technology. For example, the bicycle 12 may be of any suitable type and design, such as a conventional road bicycle or a triathlon bicycle. In various embodiments as depicted in FIGS. 4 and 5, the bicycle 12 can be a mountain bike type bicycle. In various other embodiments, the bicycle 12 is a cyclocross bicycle.

Pedal sensors 22 may be configured to measure a force exerted by the cyclist on the left and right pedals 20 a,20 b. The sensors 22 may be of any suitable type and design configured to measure the force. In one embodiment, the sensors 22 may be substantially as described in U.S. Pat. No. 8,011,242 and configured to measure radial, tangential, and other forces on the left and right pedals 20 a,20 b to facilitate observing one or more identifying features that distinguish one bodily position of the cyclist from another. The sensors 22 may be located, for example, on the left and right pedals 20 a,20 b themselves or on spindles coupled with the pedals 20 a,20 b. Such a plurality of sensors is incorporated into Garmin's Vector™ pedals, and therefore these particular pedals could function as the left and right pedals 20 a,20 b and the sensors 22. In one implementation, electronics associated with the pedals 20 a,20 b and/or the sensors 22 may be configured to perform the clip-in and clip-out event determining analysis in connection with force measurement data generated by the sensors 22. Additionally, or alternatively, the electronic device 10, may be configured to receive the force measurement data from the sensors 22 and perform this analysis to identify the one or more identifying features to determine the cyclist's bodily position. In various embodiments, sensors contained within the electronic device 10 can be used to detect movement characteristics as explained below. In various embodiments, foot pods 21 of FIGS. 4 and 5 provide sensor data to detect the movement characteristics as is also explained in connection with those figures.

As indicated, FIG. 2 is a plan view of the electronic device of FIG. 1. In various embodiments, the electronic device 10 is a bicycle computer “head” such as that provided in connection with the Garmin Edge® line of bicycle computer products. In various embodiments, the head itself contains several sensors such as accelerometers to measure static and mobile acceleration forces, gyroscopes to measure rotational motion, and barometers to measure air pressure and determine changes in altitude. It is understood that the electronic device 10 could be implemented by way of a smart device such as a smart phone or other types of wearable smart devices. In various embodiments, it is advantageous to mount the electronic device 10 to the handlebars 18 of the bicycle 12 so that the cyclist can observe and interact with the display 36 and the user interface 38 as well as because sensors associated with the electronic device 10 are able to measure parameters regarding movement of the bicycle with reference to the handlebars 18.

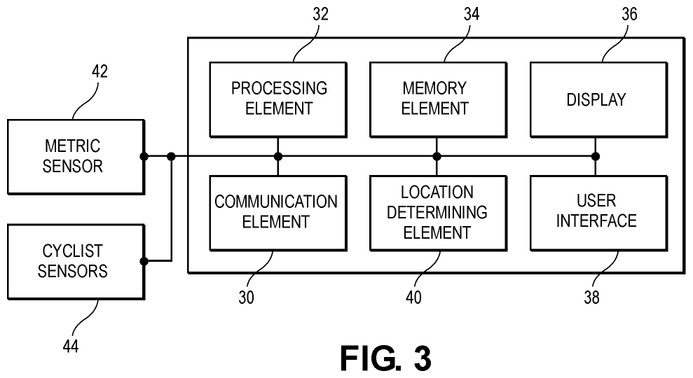

FIG. 3 is a schematic depiction of elements of the electronic device of FIG. 1. In embodiments for which the electronic device 10 both performs the analysis and communicates the results to a user, the electronic device 10 may include a communication element 30, a processing element 32, a memory element 34, and a display 36. The communication element 30 may be in wireless or wired communication with bicycle sensors 42, such as the pedal sensors 22 and configured to receive the force measurement data generated by the pedal sensors 22 regarding the force applied by the cyclist to the left and right pedals 20 a,20 b. In various embodiments, information from cyclist sensors 44 is additionally collected. The cyclist sensors 44 include heart-rate monitors and other sensors for determining the physical state of the cyclist. In configurations, the cyclist sensors 44 may include motion, acceleration, position, attitude, and other sensors configured to determine the status of the cyclist. For example, in some configurations, the cyclist sensors 44 may include a wearable chest strap that includes heart rate sensors and accelerometers to measure wearer (cyclist) cadence, torso angle, acceleration, speed, combinations thereof, and the like. Such information may be useful, as explained below, in determining the status of the cyclist. In one example, the cyclist sensors 44 include a Garmin HRM-RUN monitor.

The communication element 30 may be implemented using any appropriate technology and design, and may include signal or data transmitting and receiving circuits, such as amplifiers, filters, mixers, oscillators, digital signal processors (DSPs), and the like. Furthermore, the communication element 30 may establish communication wirelessly by utilizing radio frequency (RF) signals and/or data that comply with communication standards such as cellular 2G, 3G, or 4G, Institute of Electrical and Electronics Engineers (IEEE) 802.11 standard such as WiFi, IEEE 802.16 standard such as WiMAX, Bluetooth™, or combinations thereof. In addition, the communication element 30 may utilize communication standards such as ANT, ANT+, Bluetooth™ low energy (BLE), the industrial, scientific, and medical (ISM) band at 2.4 gigahertz (GHz), or the like. Additionally or alternatively, the communication element 30 may establish communication through connectors or couplers that receive metal conductor wires or cables or optical fiber cables.

The processing element 32 may be configured to analyze received force measurement data from the sensor 22 to identify a feature of the data which is indicative of the clip-in and clip-out events. As noted, the processing element can also receive bicycle sensor information from other sensors in communication with the electronic device 10. The processing element 32 may be implemented using any appropriate technology and design, and may include processors, microprocessors, microcontrollers, digital signal processors (DSPs), field-programmable gate arrays (FPGAs), analog and/or digital application-specific integrated circuits (ASICs), or the like, or combinations thereof. The processing element 32 may generally execute, process, or run instructions, code, code segments, software, firmware, programs, applications, apps, processes, services, daemons, or the like, or may step through the states of a finite-state machine.

The memory element 34 may be configured to record the results of the processing element's analysis over time as a series of clip-in and clip-out events while the cyclist is riding the bicycle 12. The memory element 34 may be implemented using any appropriate technology and design, and may include data storage components such as read-only memory (ROM), programmable ROM, erasable programmable ROM, random-access memory (RAM), hard disks, floppy disks, optical disks, flash memory, thumb drives, universal serial bus (USB) drives, or the like, or combinations thereof. The memory element 34 may include, or may constitute, a “computer-readable medium.” The memory element 34 may store instructions, code, code segments, software, firmware, programs, applications, apps, services, daemons, or the like that are executed by the processing element 32. The memory element 34 may also store settings, data, documents, sound files, photographs, movies, images, databases, and the like.

In embodiments, the memory element 34 may be integrated within the electronic device 10. However, in other configurations, the memory element 34 may additionally or alternatively be integrated within other components of the system, such as pedal sensors 22 and/or cyclist sensors 44. Such functionality enables functionality to be provided by these other devices (e.g., pedal sensors 22) without relying on the electronic device 10. For instance, sensors 22 and/or 44 may store performance information for the duration of a ride or lap, and that performance information can be selectively transferred after the completion of the ride or lap to the electronic device 10 or another device such as a server or cloud service accessed through communications element 30. Such functionality may allow a more comprehensive analysis of the rider performance generally, and specifically the clip-in/clip-out events described herein. This use of post-ride data transfer can enable analysis to operate outside the bounds of real-time computation, causality, and data bandwidth limitations and can noticeably improve the accuracy of the clip-in/clip-out detection.

The display 36 may be configured to present the results of the processing element's analysis of the data in real-time or, for data stored in the memory element 34, at a later time. The display 36 may be implemented using any appropriate technology and design, such as light-emitting diode (LED), organic LED (OLED), Light Emitting Polymer (LEP) or Polymer LED (PLED), liquid crystal display (LCD), thin film transistor (TFT) LCD, LED side-lit or back-lit LCD, or the like, or combinations thereof. Furthermore, the display 36 may have a round, circular, or oval shape; may possess a square or a rectangular aspect ratio which may be viewed in either a landscape or a portrait mode; and may further include a lens or other covering overlying all or part of the display 36 and configured to enhance the visibility of the information shown on the display 36.

In one implementation, the electronic device 10 may further include a user interface 38 configured to allow the cyclist or other user of the electronic device 10 to provide input regarding, for example, how the analysis is performed or how the results are displayed. The user interface 38 may be implemented using any appropriate technology and design, such as pushbuttons, rotating knobs, or the like, or combinations thereof. Furthermore, the user interface 38 may take the form of a touchscreen occupying part or all of the display 36 and allowing the user to interact with the electronic device 10 by physically touching, swiping, or gesturing on or near areas of the display 36. Additionally or alternatively, the user interface 38 may employ voice control technology or facial recognition technology.

In various embodiments, the electronic device 10 may further include or otherwise be in communication with via the communication element 30 a location-determining element 40 configured to determine a geographic location of the cyclist while the cyclist is riding the bicycle. In this embodiment, the memory element 34 may be further configured to record a series of changes in the geographic location, the processing element 32 may be further configured to correlate the series of changes in the cyclist's clip-in and clip-out status with the series of changes in the geographic location, and the display 36 may be further configured to graphically communicate the correlated series of changes in the cyclist's clip-in and clip-out status and the series of changes in the geographic location. Exemplary electronic devices having such location-determining capability include Garmin's Edge® and Forerunner® GPS devices, and therefore either of these particular devices could be configured to function as the electronic device 10.

More broadly, the location-determining element 40 may be implemented using any appropriate technology and design, such as receiving and processing radio frequency (RF) signals from a global navigation satellite system (GNSS) such as the global positioning system (GPS) primarily used in the United States, the GLONASS system primarily used in Russia and countries associated with the former Soviet Union, the BeiDou system primarily used in China, or the Galileo system primarily used in Europe. The location-determining element 40 may accompany or include an antenna to assist in receiving the satellite signals. The antenna may be a patch antenna, a linear antenna, or any other type of antenna that may be used with location or navigation devices. The location-determining element 40 may include satellite navigation receivers, processors, controllers, other computing devices, or combinations thereof, and memory. The location-determining element 40 may process a signal, referred to herein as a “location signal”, from one or more satellites that includes data from which geographic information such as the current geolocation is derived. The current geolocation may include coordinates, such as the latitude and longitude, of the current location of the electronic device 10.

It will be appreciated that substantially any other location-determining technology may be used. For example, cellular towers or any customized transmitting radio frequency towers may be used instead of satellites may be used to determine the location of the electronic device 10 by receiving data from at least three transmitting locations and then performing basic triangulation calculations to determine the relative position of the device with respect to the transmitting locations. With such a configuration, any standard geometric triangulation algorithm may be used to determine the location of the electronic device 10. The location-determining element 40 may also include or be coupled with a pedometer, accelerometer, compass, or other dead-reckoning components which allow it to determine the location of the electronic device 10. The location-determining element 40 may determine the current geographic location through a communications network, such as by using Assisted GPS (A-GPS), or from another electronic device. The location-determining element 40 may even receive location data directly from the user.

FIG. 4 is a side elevation view of the cyclist with the cyclist's left shoe clipped-in to the left pedal of the bicycle and the cyclist's right shoe clipped-out. In various embodiments, the bicycle 12 is a mountain bike-type bicycle as depicted in FIGS. 4 and 5. In this embodiment, handlebars 18′ are mountain bike-type handlebars to which the electronic device 10 is mounted. As shown in FIG. 4, the cyclist is riding with his or her left foot clipped into the left pedal and his or her right foot clipped out. It is understood that this scenario can occur in a mountain bike ride or cyclocross race when the cyclist may need to balance or push off with one or both feet.

FIG. 5 is a side elevation view of the cyclist with the cyclist's feet clipped out and walking beside the bicycle. As depicted in FIG. 5, the rider has clipped out with both feet and is walking beside the bicycle 12. In various embodiments, the electronic device 10 can identify the clip out and walk scenario by detecting signatures associated with data collected from various sensors that indicate that the cyclist has clipped out (typically with both feet) and swung a leg over the frame of the bicycle. In various embodiments, this can be detected by reference to tilt movements of the handlebars, as well as motion detection of other sensors, such as the foot pods 21 of FIGS. 4 and 5 and/or the cyclist sensors 44 described herein. The clip out and walk scenario can occur for various reasons including cyclist fatigue or terrain that is too rough to navigate while pedaling. As depicted in FIG. 5, the cyclist is walking beside the bicycle, but it is understood that the cyclist could also be carrying the bicycle, for example, with the frame over his or her shoulder, and/or to either side of the bicycle frame.

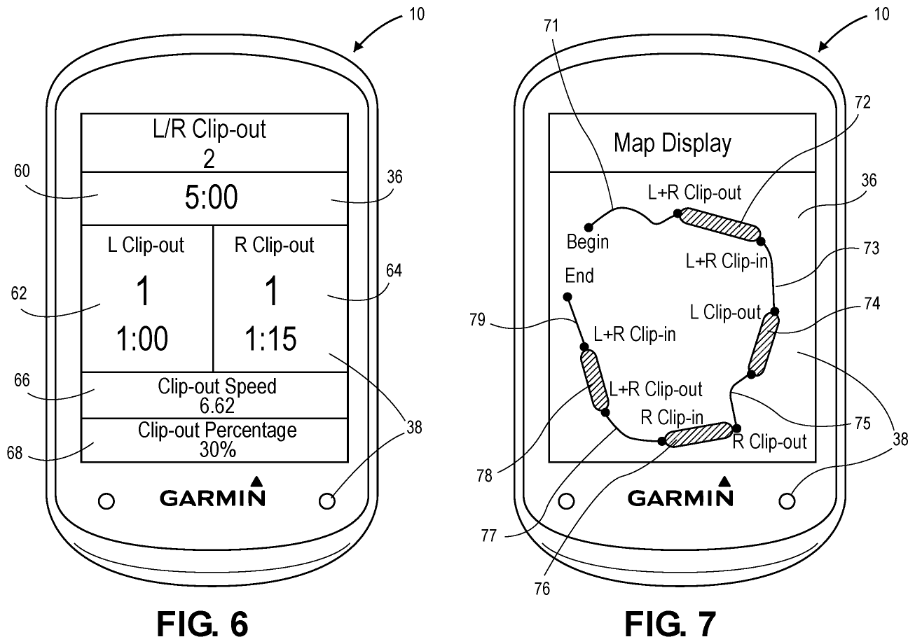

FIG. 6 is a representation of a display of clip-in and clip-out events and statistics on the electronic device 10. In various embodiments at the start of a lap or ride, a time and/or distance counter is reset to zero. From there, statistics can be collected regarding bicycle movement characteristics and clip-out events. In an embodiment, a number of clip-out events is accumulated by way of a counter and displayed to the user at segment 60 of the display 36. In this embodiment, the number of clip out events are displayed as well as the time of the last event. It is understood that by tapping, swiping or making other gestures, a user of the interface can change the time of the last clip-out event to the distance at which the last clip-out event occurred or, for example, by swiping left and right to scroll through the various clip-out events in the lap or route. In various embodiments, the electronic device accumulates statistics about left and right clip-out events separately and maintains statistical information regarding these separate events. As shown in connection with user interface elements 62 and 64, a number of left and right clip-out events are cataloged and displayed separately along with a time and or distance of occurrence or the total amount of time the cyclist's foot is clipped out. For example, as shown in FIG. 6, there has been one left clip-out event and one right clip-out event. They left event lasted for one minute, while the right event lasted for 1:15, meaning that the cyclist clipped back in first with the left foot approximately fifteen seconds before clipping back in with the right foot.

In various embodiments, it is also measured at what speed a particular clip-out occurred, and this information us correlated with other parameters and displayed to the user as shown in connection with user interface element 66. This can be a current, maximum, minimum, or average speed associated with a particular clip-out event. Additionally, the percentage of time or distance that a rider is clipped-out can provide useful information. To this end, a clip-out percentage is displayed in various embodiments, as shown in user interface element 68.

FIG. 7 is a representation of a map display of clip-in and clip-out events on a particular course. In addition to statistical information about clip-in and clip-out events, it is beneficial to the user to have graphical information about clipped-in and clipped-out statuses as correlated to the course or lap of a cyclocross race, for example. As shown in FIG. 7, this can take the form of a map display of a route on which clipped in segments (as represented by odd numbered segments, 71-79 are distinguished from either both-foot clipped out segments (72 and 78), where the cyclist would typically be walking the bike, and either left or right single-foot clip-out segments, such as left clip out segment 74 and right clip-out segment 76. These various clipped-in and clipped out segments can be identified by color, for example black when both feet are clipped in, blue when the left foot is clipped out, green when the right foot is clipped out and red when both feet are clipped out. It is understood that other color, grayscale, or cross-hatching schemes can be deployed without departing from the scope of the present teachings.

In an embodiment, the clip-in, clip-out segments of the current lap or course can be graphically compared, overlaid, or mashed-up with past laps or course of the cyclist or of other riders. These overlays can be combined with heatmaps to show areas where the cyclist (or another rider) has had to clip out in the past. In this way, a user of the electronic device 10 can evaluate the cyclist's relative performance on the current lap or course.

In another embodiment, the electronic device 10 may further include or otherwise be in communication with via the communication element 30 one or more performance metric sensors 42 configured to measure one or more aspects of the bicycle's or the cyclist's performance. These aspects may be objective or subjective, and may include any one or more of a speed of the bicycle 12, an attitude of the bicycle 12, a power output of the cyclist, a total energy used by the cyclist, a heart rate of the cyclist, an exertion perceived by the cyclist, a fatigue level of the cyclist, and a cadence of the cyclist whether walking or pedaling. In this embodiment, the memory element 34 may be further configured to record a series of changes in bicycle movement characteristics such as clip-in and clip-in and clip-out status, and one or more actual bicycle performance metrics, the processing element 32 may be further configured to correlate the series of changes in the bicycle movement characteristics with the series of changes in the one or more bicycle performance metrics, and the display 36 may be further configured to graphically communicate the correlated series of changes in the cyclist's bodily position and the series of changes in the one or more performance metrics.

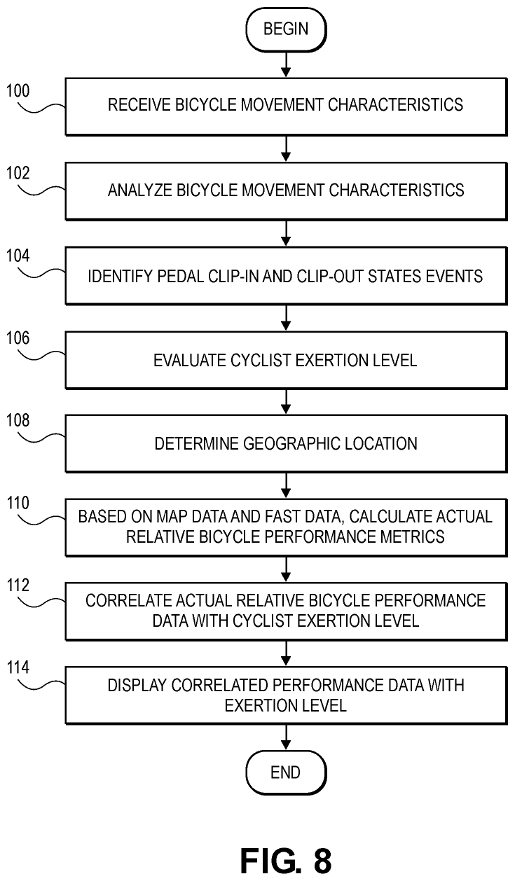

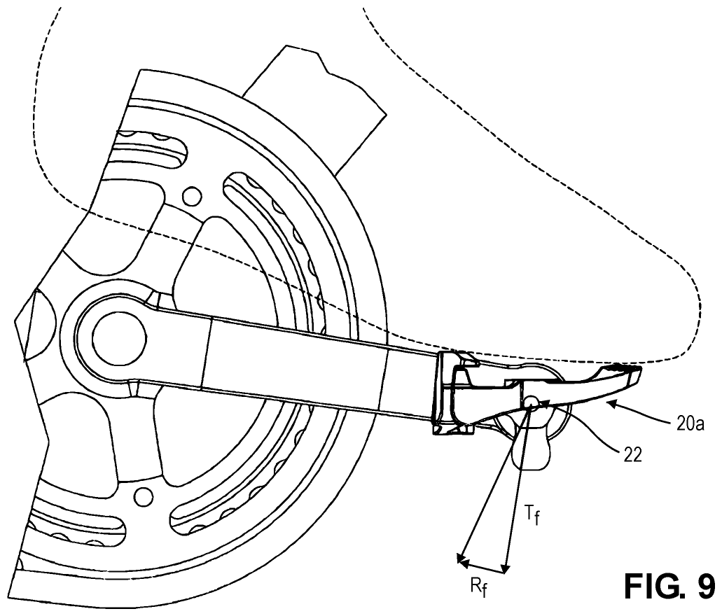

FIG. 8 is a flowchart of steps involved in a method implemented by the electronic device of FIG. 1. In operation, the electronic device 10 may function substantially as follows to determine relative cyclist performance while the cyclist is riding the bicycle 12. Broadly, the communication element 30 of the electronic device 10 may receive sensor data from the one or more bicycle sensors 42, such as pedal sensors 22, sensors associated with foot pods 21, or other sensors such as sensors internal to the electronic device 10. These sensors 42 provide information regarding bicycle movement characteristics as shown in step 100. The sensor data may be provided to the processing element 32 which analyzes it to identify signature characteristics of the data, as shown in stem 102. Various signature characteristics are indicative of events such as clip-in and clip-out events which are identified by the processing element 32, as shown in step 104. The result of the processing element's 32 analysis can be used to evaluate the cyclist's relative exertion level, as shown in step 106. For example, it is understood that on an ascent in a mountain bike ride or a cyclocross race, a cyclist will pedal uphill as long as physically practicable and will clip out only then when sufficient relative fatigue of the cyclist requires it.

With respect to repeated laps of a cyclocross race or repeated iterations of a mountain bike ride, a cyclist will have changing relative actual performance on the lap or course. Where the cyclist became sufficiently fatigued to have to clip out on a particular lap or course provides valuable information regarding the evolution of performance improvement or decline either in a particular training regime or in the midst of a cyclocross race. Accordingly, correlations of position information with exertion events, such as clipping-out and walking provide valuable information to the cyclist.

In various embodiments, the location-determining element 40 of the electronic device 10 may determine the geographic location of the cyclist, as shown in step 108. The geographic location data may be provided to the memory element 34 to be stored as a series of changes in the geographic location of the cyclist while the cyclist is riding the bicycle 12. As shown in step 110, actual relative bicycle performance metrics are calculated based on map data and past ride data (such as the cyclist's previous rides or laps or other riders' data) in connection with the geographic location data of the previous step. The processing element 32 may then correlate the actual relative bicycle performance data with a cyclist exertion level as measured in connection with step 106, as shown in step 112. The correlated actual relative bicycle performance data with a cyclist exertion level and the series of changes in the geographic location may be provided to the display 36 to be graphically displayed for the user, as shown in step 114.

FIG. 9 is a fragmentary side elevation view of the cyclist's foot exerting force on a pedal component of the bicycle of FIG. 1. In an embodiment, the electronic device 10 may determine the clip-in and clip-out events based on sensor data from the pedal sensors 22. In this embodiment, the pedal sensors 22 may be configured to measure a radial force and a tangential force applied by the cyclist to the left and right pedals 20 a,20 b. In this embodiment, a feature of the data which is indicative of clip-in and clip-out events is that the radial force Rf is higher than the tangential force Tf when the cyclist is standing or applying force at the pedal to clip in. Moreover, when the cyclist is clipped-out, effectively no forces Rf/Tf are applied to the spindle of the pedal. In another embodiment, the pedal sensors 22 may be configured to measure a clip-in or clip-out impulse created the cyclist at the left and/or right pedals 20 a,20 b when the cyclist's foot is twisted to clip-out or when the cyclist steps down with force to clip in. In this embodiment, a feature of the data which is indicative of the cyclist's clip-in status is that the total vertical force on the left and right pedals 20 a,20 b is at a higher level when the cyclist is clipping in, and the total vertical force on the left and right pedals 20 a,20 b is at a lower level when the cyclist is clipped out (after the impulse of the twisting clip-out force).

Although the technology has been described with reference to the embodiments illustrated in the attached drawing figures, it is noted that equivalents may be employed and substitutions made herein without departing from the scope of the technology as recited in the claims.

Having thus described various embodiments of the technology, what is claimed as new and desired to be protected by Letters Patent includes the following:

Although the foregoing text sets forth a detailed description of numerous different embodiments, it is understood that the detailed description is to be construed as exemplary only and does not describe every possible embodiment because describing every possible embodiment would be impractical, if not impossible. In light of the foregoing text, numerous alternative embodiments may be implemented, using either current technology or technology developed after the filing date of this patent application.

What is claimed is:

1. An electronic device for use in connection with a bicycle having handlebars and left and right pedals, the electronic device configured to determine and communicate to a user of the device an effort level exerted by a cyclist and a corresponding bicycle performance metric achieved by the cyclist based on movement characteristics of the bicycle and whether one or both of the cyclist's shoes are clipped into the pedals, the device comprising:
at least one sensor for determining the movement characteristics of the bicycle;
at least one memory element configured to store the movement characteristics of the bicycle;
at least one processing element coupled with the sensor and the memory element, the processing element configured to:
ascertain at least one pedal clip status event based on the movement characteristics of the bicycle; and
display information about the effort level exerted by the cyclist and the corresponding bicycle performance metric.

2. The electronic device of claim 1, wherein the pedal clip status events comprise:
clipping into a pedal by one or both feet;
clipping out of a pedal by one or both feet; and
swinging a leg of the cyclist over the bicycle frame.

3. The electronic device of claim 2, wherein, based on the pedal clip status events, the processing element is further configured to distinguish between activity modes comprising:
active riding mode, characterized in that the cyclist is riding the bicycle with one or both shoes clipped in;
straddle stopped mode, characterized in that the cyclist and the bicycle are stopped with the cyclist straddling the frame of the bicycle with one or both shoes clipped out;
straddle walking mode, characterized in that the cyclist is walking the bike with one or both feet clipped out; and
side walking mode, characterized in that the cyclist is walking the bike with both feet clipped out, having dismounted and swung a leg over the frame of the bike.

4. The electronic device of claim 1, wherein the accelerometer is located within a head component of a bicycle computer mounted on the handlebars of the bicycle.

5. The electronic device of claim 1, wherein the at least one sensor is a wearable sensor worn on the body of the cyclist.

6. The electronic device of claim 1, wherein the at least one sensor further comprises a force detecting sensor in the spindle of the pedals.

7. The electronic device of claim 1, wherein the sensor further comprises a GPS unit.

8. The electronic device of claim 1, wherein the performance metric is based on performance factors comprising:
a pedal cadence;
and
a forward progress metric.

9. The electronic device of claim 8, wherein the forward progress metric is determined by forward progress factors comprising:
acceleration and velocity measured at the accelerometer;
relative altitude changes as measured in connection with a barometer associated with the electronic device; and
location characteristics as measured by a GPS unit and corresponding map information stored in the memory.

10. The electronic device of claim 8, wherein the forward progress metric further comprises a relative progress metric comparing performance in the current ride with corresponding metrics of previous rides or other riders.

11. The electronic device of claim 10, wherein the relative progress metric further comprises a heat map regarding a difficulty associated with a segment of the course being ridden.

12. An electronic device for use in connection with a bicycle having at least one gear selector to determine a selected gear in which the bicycle is being operated, handlebars, and left and right pedals, the electronic device configured to determine and communicate to a user of the device an effort level exerted by a cyclist and a corresponding bicycle performance metric achieved by the cyclist based on movement characteristics of the bicycle and whether one or both of the cyclist's shoes are clipped into the pedals, the device comprising:
at least one sensor for determining the movement characteristics of the bicycle;
at least one memory element configured to store the movement characteristics of the bicycle;
at least one processing element coupled with the sensor and the memory element, the processing element configured to:
ascertain at least one pedal clip status event based on the movement characteristics of the bicycle; and
display information about the effort level exerted by the cyclist and the corresponding bicycle performance metric, wherein the bicycle performance metric is based on performance factors comprising:
a pedal cadence;
the selected gear; and
a forward progress metric.

13. The electronic device of claim 12, wherein the pedal clip status events comprise:
clipping into a pedal by the right foot at a right clip-in rate;
clipping into a pedal by the left foot at a left clip-in rate;
clipping out of a pedal by the right foot at a right clip-out rate;
clipping out of a pedal by the left foot at a left clip-out rate; and
swinging a leg of the cyclist over the bicycle frame.

14. The electronic device of claim 13, wherein, based on the pedal clip status events, the processing element is further configured to distinguish between activity modes comprising:
active riding mode, characterized in that the cyclist is riding the bicycle with one or both shoes clipped in;
straddle stopped mode, characterized in that the cyclist and the bicycle are stopped with the cyclist straddling the frame of the bicycle with one or both shoes clipped out;
straddle walking mode, characterized in that the cyclist is walking the bike with one or both feet clipped out; and
side walking mode, characterized in that the cyclist is walking the bike with both feet clipped out, having dismounted and swung a leg over the frame of the bike.

15. The electronic device of claim 12, wherein the accelerometer is located within a head component of a bicycle computer mounted on the handlebars of the bicycle.

16. The electronic device of claim 12, wherein the at least one sensor is a wearable sensor worn on the body of the cyclist.

17. The electronic device of claim 12, wherein the at least one sensor further comprises a force detecting sensor in the spindle of the pedals.

18. An electronic device for use in connection with a bicycle having at least one gear selector to determine a selected gear in which the bicycle is being operated, handlebars, and left and right pedals, the electronic device configured to determine and communicate to a user of the device an effort level exerted and a corresponding performance metric achieved by a cyclist based on movement characteristics of the bicycle and whether one or both of the cyclist's shoes are clipped into the pedals, the device comprising:
at least one sensor for determining the movement characteristics of the bicycle;
at least one memory element configured to store the movement characteristics of the bicycle;
at least one processing element coupled with the sensor and the memory element, the processing element configured to:
ascertain at least one pedal clip status event based on the movement characteristics of the bicycle, wherein the pedal clip status events comprise clipping into a pedal by one or both feet; and
display information about the effort level exerted by the cyclist and the corresponding performance metric, wherein the performance metric is based on performance factors comprising:
a pedal cadence;
the selected gear; and
a forward progress metric.

19. The electronic device of claim 18, wherein the pedal clip status events comprise:
clipping into a pedal by one or both feet;
clipping out of a pedal by one or both feet; and
swinging a leg of the cyclist over the bicycle frame, and wherein, based on the pedal clip status events, the processing element is further configured to distinguish between activity modes comprising:
active riding mode, characterized in that the cyclist is riding the bicycle with one or both shoes clipped in;
straddle stopped mode, characterized in that the cyclist and the bicycle are stopped with the cyclist straddling the frame of the bicycle with one or both shoes clipped out;
straddle walking mode, characterized in that the cyclist is walking the bike with one or both feet clipped out; and
side walking mode, characterized in that the cyclist is walking the bike with both feet clipped out, having dismounted and swung a leg over the frame of the bike.

20. The electronic device of claim 19, wherein the bicycle is of a type selected from the group of:
a mountain bike; and a cyclocross bike.
